(19) United States
(12) Patent Application Publication
(10) Pub. No.: US 2007/0067190 A1
(43) Pub. Date: Mar. 22, 2007

(54) Method And Apparatus to Provide for the Provision of Medically-Related Information

(76) Inventor: William A. Yasnoff, Arlington, VA (US)

Correspondence Address: FITCH EVEN TABIN AND FLANNERY, 120 SOUTH LA SALLE STREET, SUITE 1600, CHICAGO, IL, 60603-3406, US

(21) Appl. No.: 11/533,209

(22) Filed: Sep. 19, 2006

Related U.S. Application Data
(60) Provisional application No. 60/719,042, filed on Sep. 21, 2005.

(51) Int. Cl. G06F 19/00; A61B 5/00

(52) U.S. Cl. 705/3; 600/300

(57) ABSTRACT

One provides (102) an opportunity for an information-processing entity to submit medically-related information regarding individuals. Upon receiving (103) such information from such an information-possessing entity, one then arranges (104) for the digital storage of such medically-related information as well as corresponding compensation to be provided (105) to the information-possessing entity for having provided such information. By one approach these teachings will further accommodate receiving (101) consideration from any of a variety of entities. This consideration can serve, at least in part, as a source for the aforementioned compensation.

### RELATED APPLICATIONS

This application claims the benefit of U.S. Provisional Application No. 60/719,042, filed Sep. 21, 2005, the contents of each of which are fully incorporated herein by this reference.

### TECHNICAL FIELD

This invention relates generally to information and more particularly to medically-related information.

### BACKGROUND

For many persons living in a modern society, the development of medically-related information begins at an early age. Such information includes, by way of example and not by way of limitation, data regarding physical development and status, diseases, injuries, allergies and sensitivities, congenital conditions, and corresponding prognoses, cures, therapies, prescriptions, inoculations, treatments, operations, and so forth. Considerable agreement exists that such medically-related information comprises important (even critical) information regarding a given individual. Such information, for example, can contribute greatly to more efficiently and quickly determining a particular course of action to pursue with respect to a given individual seeking treatment or assistance.

Unfortunately, for the most part, such medically-related information tends to be dispersed in a highly disaggregated manner over various caregivers and other interested parties. Individuals can and will, over the course of a lifetime, often seek assistance from a multitude of unrelated service providers. At present, whatever records might exist for a given individual must be physically copied and provided by a first service provider to another service provider in order to provide the latter with access to such information. This disaggregated approach persists even in many clinic and shared practice settings where duplicative records for a given individual are likely to exist to ensure that each caregiver has access to relevant information for their patients.

That establishment and maintenance of a universal health information infrastructure represents a consummation devoutly to be wished, in and of itself, unfortunately does not make it so. At present, at least one enormous stumbling block in this regard comprises founding such a program on a financially sustainable basis. Some studies have indicated that caregivers such as physicians might be required to make an initial investment of $40,000 or more in order to be able to reliably and efficiently participate in providing information regarding their patients. Unfortunately, however, some studies also indicate that nearly 90% of the benefits of such a system will go to someone other than these primary caregivers.

That a centralized store of medically-related information represents a generally good idea seems clear. Attempting to found such a system merely upon the altruism of primary caregivers, however, seems just as clearly to be an unsuccessful approach.

### BRIEF DESCRIPTION OF THE DRAWINGS

The above needs are at least partially met through provision of the method and apparatus to provide for the provision of medically-related information described in the following detailed description, particularly when studied in conjunction with the drawings, wherein:

FIG. 1 comprises a flow diagram as configured in accordance with various embodiments of the invention;

FIG. 2 comprises a block diagram as configured in accordance with various embodiments of the invention; and

FIG. 3 comprises a block diagram as configured in accordance with various embodiments of the invention.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. It will further be appreciated that certain actions and/or steps may be described or depicted in a particular order of occurrence while those skilled in the art will understand that such specificity with respect to sequence is not actually required. It will also be understood that the terms and expressions used herein have the ordinary meaning as is accorded to such terms and expressions with respect to their corresponding respective areas of inquiry and study except where specific meanings have otherwise been set forth herein.

### DETAILED DESCRIPTION

Generally speaking, pursuant to these various embodiments, one provides an opportunity for an information-processing entity to submit medically-related information regarding individuals. Upon receiving such information from such an information-possessing entity, one then arranges for the digital storage of such medically-related information as well as corresponding compensation to be provided to the information-possessing entity for having provided such information. By one approach these teachings will further accommodate receiving consideration from any of a variety of entities. This consideration can serve, at least in part, as a source for the aforementioned compensation.

By compensating information-possessing entities for submitting such medically-related information, a means now exists for economically motivating parties such as primary health care providers to make the investment and the effort to submit such information regarding their patients. This, in turn, provides a working basis upon which an initial store of medically-related information can be established and then, going forward, can be maintained. These teachings, therefore, provide a means of facilitating the establishment of the presently desired universal health information infrastructure that otherwise seems to only be an impossible dream.

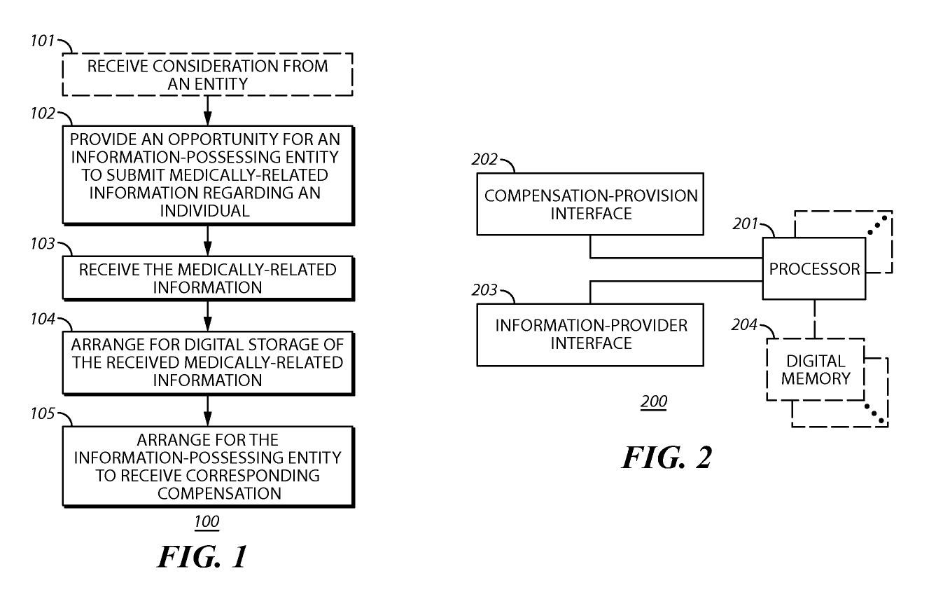

These and other benefits may become clearer upon making a thorough review and study of the following detailed description. Referring now to the drawings, and in particular to FIG. 1, a process 100 illustrative of these teachings provides 102 an opportunity for an information-possessing entity to submit medically-related information regarding at least a first individual. This opportunity can be extended in any number of ways. By one approach, this opportunity can be extended using an extranet-based opportunity (such as, but not limited to, an Internet-based opportunity).

The specifics of the opportunity can also vary with respect to the needs and/or opportunities presented by a given application setting. For example, by one approach, this opportunity can include the opportunity to submit such information in a facsimile format. As another example, this opportunity can include the opportunity to submit such information using a browser-based opportunity. The mechanics of such opportunities are generally well understood in the art. As the present teachings are not particularly sensitive regarding the selection of any present approach in this regard, for the sake of brevity further elaboration in this regard will not be presented here.

These teachings will readily accommodate extending such an opportunity to a relatively wide audience of candidate information-possessing entities. This can certainly include medical services providers, including but not limited to all manner of caregivers such as doctors, nurses, emergency rooms and other urgent care facilities, laboratory technicians, diagnosticians, and so forth. Other potentially relevant entities include, but are not limited to, the individual themselves, a provider of medical insurance services, a provider of pharmacological services, a prescription processing clearinghouse, a provider of long-term care, a provider of home health care, a provider of ambulance services, a provider of emergency medical services, a provider of disease management services, a provider of hospice services, and so forth. Those skilled in the art will recognize and appreciate that these teachings are also likely to apply to and encompass future services and service providers as may arise in the future.

In a somewhat similar manner, these teachings will also readily accommodate extending this opportunity to invite the submission of a relatively wide range of medically-related information. Relevant examples include, but are certainly not limited to, diagnostic information, treatment-related information, prescription-related information, patient-based information, accident information, injury information, medical services provider information, medical history information, medical outcomes information, and so forth as well as updates and/or corrections to existing or previously submitted medically-related information.

At some point, one might hope that essentially all information for all individuals might be receivable via such an opportunity. Nevertheless, to accommodate important notions of privacy, such an opportunity can be conditioned as appropriate to permit or invite, for example, only medically-related information for individuals who have authorized provision of such medically-related information. By one approach, a given individual may pre-authorize the submission of such information on their behalf.

By another approach, this opportunity may allow such permission to accompany submitted information. By yet another approach, this opportunity may permit such a submission in the absence of such permission having been obtained, followed by attainment of that permission.

This process 100 then provides for receiving 103 medically-related information from an information-possessing entity for a given individual in response to the aforementioned opportunity. Such information can be received via, for example, an extranet-based approach such as an Internet-facilitated submission facility. By one approach, such information can be submitted on a batch basis. So configured, a batch transfer of informational content as pertains to a single individual and/or a plurality of individuals can be transmitted on, for example, a daily or weekly basis. By another approach, such information could be submitted as discrete transfers of informational content as pertain, for example, to only a single individual and/or reporting event.

As noted, the receipt of such medically-related information can be conditioned, if desired, upon the individual's permission, authorization, and consent. Such receipt may also be conditioned upon other criteria if so desired. For example, by one approach, such information may be refused when the received information is duplicative of previously received information. In the alternative, if desired, all such information might be accepted upon receipt notwithstanding partial or complete duplicity. As another example, by another approach, such information may be refused when the received information is stale. This might be the case, for example, when the information corresponds to an individual who has since died. As yet another example, by yet another approach, such information may be refused when the received information is of a non-useful type or content.

This process 100 then arranges 104 for the digital storage of the received medically-related information. This can be accomplished in any number of ways. As will be well understood by one skilled in the art, this can comprise arranging for digital storage of the received medically-related information at a central repository. In the alternative, and/or in combination with a centralized approach, this can also comprise arranging for digital storage of the received medically-related information at a plurality of distributed storage locations. By one approach, the complete body of information can be stored at each such distributed storage location. By another approach, the information itself can be parsed over various ones of such a plurality of distributed storage locations.

This process 100 then arranges 105 for the information-possessing entity to receive compensation in exchange for having provided the medically-related information. Such compensation can comprise monetary and/or non-monetary forms of compensation as may best meet the needs and/or requirements of a given application setting. Such an arrangement can be effected in any of a variety of ways. By one approach this can comprise arranging for a substantially immediate transaction. By other approaches, this can comprise arranging for, for example, periodic transactions to effect such compensation, a per-event transaction such that the information-possessing entity receives such compensation with each such information submission event, and/or a batched-event transaction where the information-possessing entity receives compensation as corresponds to any number of submissions and/or any number of corresponding individuals over, for example, a given period of time.

Those skilled in the art will further appreciate that this compensation mechanism also allows for at least some control or leveraging of the formats and standards that are used for the submission of such information by these various parties. For example, instead of be required to accept such information in whatever form might be convenient to the information provider, the receiving party can instead condition all or part of the compensation upon a requirement that the information be provided in a particular way. This can include an order by which such information is presented, specific items of required information, and/or a communication medium or bearer format by which such information is provided. This, in turn, can greatly simplify the task of the receiving party and/or another party seeking to use and leverage that information to make quick and efficient use of that information as interpretation, reformatting, and other similar processing tasks may be partially or completely avoided. Absent such compensation, information providers have no particular incentive to make their information compatible with processing needs outside their own work environment. These teachings are therefore seen to greatly facilitates interoperability as well as efficient use of such information.

To provide at least a partial basis for such compensatory events, this process 100 will also optionally provide for receiving 101 consideration from one or more entities such as, but not limited to, the individuals themselves, an employer of one or more such individuals, a medical services benefits provider, a medical services benefits administrator, a governmental entity, and so forth. Any number of consideration collection mechanisms could serve in this regard. As but one simple illustration in this regard, individuals interested in having their medically-related information collected and maintained as per these teachings could make a modest monthly payment to support these activities. As another simple illustration in this regard, medical insurance coverage providers could pay an annual amount on a pro-rated scale that is commensurate with their customer population size.

So configured, such a process provides a sustainable basis for receiving initial and ongoing funding from parties having an interest in creating, maintaining, and/or benefiting from a comprehensive store of medically-related information for individuals and for providing self-interested motivation and means to parties having medically-related information to submit such information for such purposes. Such a process can be executed in tight conjunction with the actual receipt and storage of such information or can be implemented in a more removed fashion if desired. It will be understood that these teachings could be implemented by either a for-profit or a not-for-profit entity.

Those skilled in the art will appreciate that the above-described processes are readily enabled using any of a wide variety of available and/or readily configured platforms, including partially or wholly programmable platforms as are known in the art or dedicated purpose platforms as may be desired for some applications. Referring now to FIG. 2, an illustrative approach to such a platform will now be provided.

In this example the apparatus 200 comprises a processor 201 that operably couples to a compensation-provision interface 202 and an information-provider interface 203. The processor 201 can comprise, for example, a server platform that is partially or wholly programmable. In such a case, the processor 201 can be readily programmed to operate in accordance with the teachings set forth herein. For example, the processor 201 can be configured and arranged to provide an opportunity (via the information-provider interface 203) for an information-possessing entity to submit medically-related information regarding at least a first individual, to receive (again via the information-provider interface) medically-related information proffered by such an information-possessing entity, and to arrange, using the compensation-provision interface 203, for the information-possessing entity to receive compensation in exchange for the information-possessing entity having provided the medically-related information.

Such a processor 201 can also be configured and arranged to arrange for digital storage of proffered medically-related information at one or more digital memories 204. If desired, such a digital memory 204 can also be employed to store information regarding consideration that has been received (as described above) to facilitate offsetting, at least in part, the compensation that is being offered and provided to such information-possession entities.

Those skilled in the art will recognize and understand that such an apparatus 200 may be comprised of a plurality of physically distinct elements as is suggested by the illustration shown in FIG. 2. It is also possible, however, to view this illustration as comprising a logical view, in which case one or more of these elements can be enabled and realized via a shared platform. It will also be understood that such a shared platform may comprise a wholly or at least partially programmable platform as are known in the art. As but one example in this regard, the compensation-provision interface 202 and/or the information-provider interface 203 can comprise an integral part of the processor 201 (as when, for example, these interfaces comprise browser-based interfaces that comprise, in turn, a part of a domain that is hosted by the processor 201).

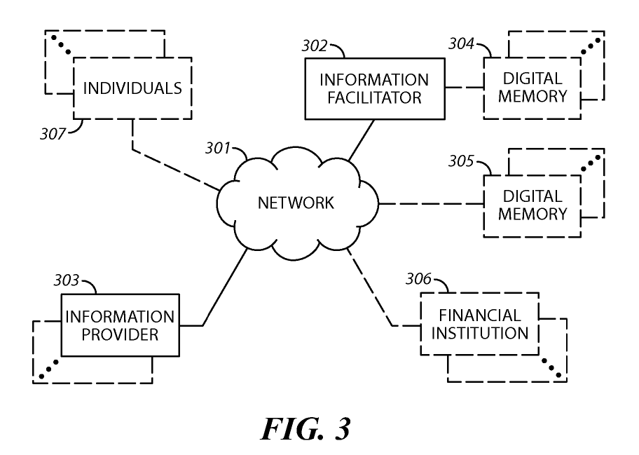

Those skilled in the art will recognize and appreciate that these teachings can be readily leveraged in a variety of ways and are highly scalable as well. Such flexibility, in turn, offers a considerable concrete advantage with respect to effectively fielding a viable health information infrastructure that will offer adequate behavioral inducements to effect the sought for tangible results. With reference to FIG. 3, for example, a network 301 such as the Internet can serve to readily link an information facilitator 302 that operates in accordance with the teachings set forth herein to prompt the submission of medically-related information from a variety of information providers 303 as pertains to a subject base of individuals 307. By one approach, if desired, the information facilitator 302 can itself tend to the digital storage of such proffered information using one or more corresponding digital memories 304. By another approach, if desired, the information facilitator 302 can instead facilitate the storage of such information using one or more remotely located and/or controlled digital memories 305. Accordingly, for example, the information facilitator 302, while serving the role of providing the information submission opportunity and of facilitating the compensation role described above, can otherwise be as involved, or not involved, in the actual handling of the medically-related information as may be desired.

Similarly, by one approach, the information facilitator 302 can arrange, if desired, for direct compensation of the information providers as per these teachings. By another approach, however, it would also be possible for the information facilitator 302 to cooperate instead with one or more financial institutions 306 in this regard. To illustrate, by one approach the information facilitator 302 could provide compensation information (such as the identity of the entity to be compensated and a specific amount by which such entity is to be compensated) to a given financial institution 306 which in turn then effects the actual compensation step.

Those skilled in the art will recognize that a wide variety of modifications, alterations, and combinations can be made with respect to the above described embodiments without departing from the spirit and scope of the invention, and that such modifications, alterations, and combinations are to be viewed as being within the ambit of the inventive concept. For example, additional precautions could be taken to aid with detecting and/or preventing the fraudulent submission of information for the purpose of gaining unjustified compensation. By one approach, information might only be accepted from a trusted source that has been vetted in some particular manner. By another approach, information submission activities and patterns could be monitored and automatically analyzed to detect patterns and events that tend to serve as reliable indicators of potential fraudulent activity. It would also be possible, for example, to require a co-validation from a given individual before accepting a submission of medically-related information for that individual. As yet another example in this regard, it would also be possible to encyrpt the information being provided to thereby further facilitate the secure and private handling of such information.

I claim:

1. A method comprising:
providing an opportunity for an information-possessing entity to submit medically-related information regarding at least a first individual;
receiving the medically-related information from the information-possessing entity to provide received medically-related information;
arranging for digital storage of the received medically-related information;
arranging for the information-possessing entity to receive compensation in exchange for the information-possessing entity having provided the medically-related information.

2. The method of claim 1 wherein providing an opportunity for an information-possessing entity to submit medically-related information regarding at least a first individual comprises, at least in part, providing an extranet-based opportunity.

3. The method of claim 2 wherein the extranet-based opportunity comprises an Internet-based opportunity.

4. The method of claim 3 wherein the Internet-based opportunity comprises a browser-based opportunity.

5. The method of claim 1 wherein the information-possessing entity comprises at least one of:
a medical services provider;
the first individual;
a provider of medical insurance services;
a provider of pharmacological services;
a prescription processing clearinghouse;
a provider of long-term care;
a provider of home health care;
a provider of ambulance services;
a provider of emergency medical services;
a provider of disease management services;
a provider of hospice services.

6. The method of claim 1 wherein the medically-related information comprises at least one of:
diagnostic information;
treatment-related information;
prescription-related information;
patient-based information;
accident information;
injury information;
medical services provider information;
medical history information;
medical outcomes information;
an update to existing medically-related information;
a correction to existing medically-related information.

7. The method of claim 1 wherein receiving the medically-related information from the information-possessing entity comprises receiving the medically-related information from the information-possessing entity via an extranet.

8. The method of claim 1 wherein receiving the medically-related information from the information-possessing entity comprises receiving the medically-related information as at least one of:
a batch transfer of informational content as pertains to a plurality of individuals;
a discrete transfer of informational content as pertains only to a single individual.

9. The method of claim 1 wherein arranging for digital storage of the received medically-related information comprises arranging for digital storage of the received medically-related information at a central repository.

10. The method of claim 1 wherein arranging for digital storage of the received medically-related information comprises arranging for digital storage of the received medically-related information at a plurality of distributed storage locations.

11. The method of claim 1 wherein arranging for the information-possessing entity to receive compensation comprises arranging for the information-possessing entity to receive compensation via at least one of:
a substantially immediate transaction;
a periodic transaction;
a per-event transaction;
a batched-event transaction.

12. The method of claim 1 wherein providing an opportunity for an information-possessing entity to submit medically-related information regarding at least a first individual comprises providing an opportunity for an information-possessing entity to submit medically-related information regarding only individuals who have authorized provision of the medically-related information.

13. The method of claim 1 further comprising:
receiving consideration from an entity.

14. The method of claim 13 wherein arranging for the information-possessing entity to receive compensation in exchange for the information-possessing entity having provided the medically-related information comprises using the consideration to form, at least in part, the compensation.

15. The method of claim 13 wherein receiving consideration from an entity comprises receiving the consideration from at least one of:
the at least one individual;
an employer of the at least one individual;
a medical services benefits provider;
a medical services benefits administrator;
a governmental entity.

16. The method of claim 13 wherein the consideration comprises at least one:
monetary consideration;
non-monetary consideration.

17. An apparatus comprising:
a compensation-provision interface;
an information-provider interface;
a processor operably coupled to the digital memory, the compensation-provision interface, and the information-provider interface and being configured and arranged, at least in part, to:
provide an opportunity via the information-provider interface for an information-possessing entity to submit medically-related information regarding at least a first individual;
receive, via the information-provider interface, the medically-related information from the information-possessing entity to provide received medically-related information;
arrange for digital storage of the received medically-related information;
arrange, using the compensation-provision interface, for the information-possessing entity to receive compensation in exchange for the information-possessing entity having provided the medically-related information.

18. The apparatus of claim 17 further comprising:
a digital memory operably coupled to the processor for storing information as corresponds to the received medically-related information;
and wherein the processor is further configured and arranged to provide for digital storage of the received medically-related information, at least in part, using the digital memory.

19. The apparatus of claim 17 further comprising:
a digital memory operably coupled to the processor for storing information regarding consideration that has been received to facilitate offsetting, at least in part, the compensation being offered to the information-possession entity.
